(19) United States
(12) Patent Application Publication
(10) Pub. No.: US 2004/0006117 A1
(43) Pub. Date: Jan. 8, 2004

(54) Microglia inhibitors for interrupting interleukin 12 and IFN-gamma-mediated immune reactions

(75) Inventors: Thorsten Blume, Schildow (DE); Wolf-Dietrich Doecke, Berlin (DE); Wolfgang Halfbrodt, Berlin (DE); Joachim Kuhnke, Potsdam (DE); Ursula Moenning, Woltersdorf (DE); Bernd Elger, Berlin (DE); Herbert Schneider, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: SCHERING AKTIENGESELLSCHAFT, Berlin (DE)

(21) Appl. No.: 10/366,703

(22) Filed: Feb. 14, 2003

Related U.S. Application Data
(60) Provisional application No. 60/357,833, filed on Feb. 21, 2002.

(30) Foreign Application Priority Data
Feb. 15, 2002 (DE) 10207843.2

(51) Int. Cl. A61K 31/4184

(52) U.S. Cl. 514/394

(57) ABSTRACT

The invention describes the use of microglia inhibitors for the production of pharmaceutical agents that inhibit immune reactions that are mediated by monocytes, macrophages and T cells, and their use for treating T-cell-mediated immunological diseases and non-T-cell-mediated inflammation reactions.

This application claims the benefit of the filing date of U.S. Provisional Application Serial No. 60/357,833 filed Feb. 21, 2002.

The invention relates to the use of a microglia inhibitor for the production of a pharmaceutical agent that inhibits immune reactions that are mediated by interleukin 12 (IL 12) and interferon-γ and its use for treating T-cell-mediated immunological diseases and non-T-cell-mediated inflammation reactions.

The immune system comprises a considerable number of cells and tissue complexes that mainly communicate with one another via soluble factors. It is known that many unmunological diseases are triggered by an imbalance of soluble immune factors, such as, e.g., the cytokines (Mosmann and Coffmann., Ann. Rev. Immunol. 7: 145-173 (1989 Street and Mosmann, FASEB J. 5: 171-177 (1991); Lucey et al., Clin. Microbiol. Rev. 4: 532-562 (1996); Powrie and Coffman, Trends Pharmacol. Sci 14: 164-168 (1993); Singh et al., Immunolog. Res., 20: 164-168 (1999)). There are, for example, a considerable number of references to a role of interferon gamma and interleukin 12 in the pathogenesis of autoimmune diseases. Especially to be cited are diseases that are characterized by a T-cell-mediated inflammatory reaction, such as multiple sclerosis, diabetes, chronic inflammatory intestinal diseases (inflammatory bowel diseases). The cytokine interleukin 12 (IL 12) is produced from phagocytic cells, such as macrophages/monocytes, dendrites, B cells and other antigen-presenting cells (APC) and influences both the function of natural killer cells (NK cells) and those of T-lymphocytes. In both cell types, IL 12 can stimulate the production of interferon gamma (IFNγ). T-Lymphocytes can be divided roughly into two categories that are characterized by the expression of certain surface antigens (CD4 and CD8): CD4-positive T cells (helper T cells) and CD8-positive T cells (cytotoxic T cells). The CD4 cells can in turn be divided into T-helper cells 1 (Th 1) and T-helper cells 2 (Th 2). Especially the Th1-mediated immunological responses are associated with the pathogenesis of numerous immune diseases, especially the autoimmune diseases, such as, e.g.: type I insulin-dependent diabetes mellitus (IDDM), multiple sclerosis, allergic contact dermatitis, psoriasis, rheumatoid arthritis, chronic inflammatory intestinal diseases (“inflammatory bowel diseases”—Crohn's disease, colitis ulcerosa), lupus diseases and other collagenoses as well as acute rejection reactions in allografts (“host-versus-graft”—allograft rejection, “graft-versus-host disease”).

Of interleukin 12, it is known that it plays a critical role in the regulation of the Th1 response. In these cells, interleukin 12 induces the production of mainly IL-2, IFNγ, TNFα and TNFβ (Mosmann and Sad, Immunol. Today 17: 138-146 (1996); Gately et al., Annu. Rev. Immunol. 16: 495-521 (1998)). Especially IFNγ is a potent mediator of the IL-12 action. An over-production of interferon gamma can be responsible for, for example, the MHC II (Major Histocompatibility Complex)-associated autoimmune diseases. (In addition, there is also sufficient evidence with respect to a pathological role of interferon gamma in allergic diseases as well as sarcoidosis and psoriasis (Billiau, A., Adv. Immunol., 62: 61-130 (1996); Basham et al. J. Immunol. 130: 1492-1494 (1983); Hu et al., Immunology, 98: 379-385 (1999); Seery, J. P., Arthritis Res., 2: 437-440 (2000)). Moreover, IL-12 and IL-12/IL-18-induced IFNγ from NK cells are essentially involved in the pathomechanism of non-T-cell-mediated inflammation reactions (e.g., “Toxic Shock Syndrome,” endotoxemia, sepsis and septic shock, ARDS, “first dose response” in the case of antibody therapy, e.g., OKT3 administration in the case of allografts) (Kum et al., Infect Immun. 69: 7544-7549 (2001); Arad et al., J. Leukoc. Biol. 69: 921-927 (2001); Hultgren et al., Arthritis Res. 3: 41-47 (2001), Arndt et al., Am. J. Respir. Cell. Mol. Biol 22: 708-713 (2000); Grohmann et al., J. Immunol. 164: 4197-4203 (2000); Muraille et al., Int. Immunol. 11: 1403-1410 (1999)). IL-12 also plays a role in inflammations with pathomechanisms that are unclear at this time (e.g., eclampsia) (Hayakawa et al., J. Reprod. Immunol. 47: 121-138 (2000); Daniel et al., Am. J. Reprod. Immunol. 39: 376-380 (1998)).

In addition to interleukin 12 and IFNγ, still other cytokines are ascribed a role in the pathogenesis of immune diseases and systemic inflammation reactions, such as, for example, the TNFα. TNFα plays an important pathological role in the case of infectious diseases (such as sepsis, “toxic shock syndrome” (Tracey et al., Nature 330: 662-664 (1987); Basger et al., Circ. Shock, 27: 51-61 (1989); Hinshaw et al., Circ. Shock, 30: 279-292 (1990); Waage, A., Lancet, 351: 603 (1998); Cohen et al., Lancet, 351: 1731 (1998)), but also numerous other immune-mediated diseases.

For the treatment of IL 12-mediated diseases and for the reduction of acute symptoms of these diseases, corticosteroids are frequently used, whose side effects especially in long-term treatment often result in a termination of treatment.

The activation of microglia represents a central step in the inflammation process of almost all degenerative diseases of the central nervous system. The microglia can remain in the activated state over an extended period, in which they produce and secrete various inflammation factors, e.g., reactive oxygen/nitrogen intermediate compounds, proteases, cytokines, complement factors and neurotoxins. The latter in turn produce neuronal dysfunction and degeneration. The activation of microglia can be carried out by various stimuli such as, e.g., Aβ-peptide (β-amyloid, Araujo, D. M. and Cotman, C. M., Brain Res. 569: 141-145 (1992)), prion protein, cytokines or by cell fragments (Combs, C. K. et al., J. Neurosci. 19: 928-939 (1999); Wood, P. L., Neuroinflammation: Mechanisms and Management, Humana Press (1998).

Compounds that inhibit the activation of microglia after stimulation with the Aβ-peptide are described in WO 01/51473, DE File No. 101 34 775.8 and DE File No. 101 35 050.3. Moreover, it is also known that benzimidazoles that inhibit the activation of microglia are suitable for treatment of neuroinflammatory diseases, such as AIDS dementia, amyotrophic lateral sclerosis, Creutzfeldt-Jakob disease, Down's Syndrome, diffuse Lewy Body disease, Huntington's disease, leukencephalopathy, multiple sclerosis, Parkinson's disease, Pick's disease, Alzheimer's disease, stroke, temporary lobe epilepsy and of tumors.

The invention is based on the problem of preparing an agent that is suitable for treating immunological diseases, whereby the immunological diseases are triggered by increased production of cytokines, such as, e.g., IL 12, IFNγ, and TNFα without having an effect on other factors in the immune system, by which side effects are reduced or prevented.

It has now been found that, surprisingly enough, microglia inhibitors inhibit the production of IL 12 and IFNγ and induce the interleukin-10 production (IL 10) and therefore are used for the production of a pharmaceutical agent for treating a monocyte-mediated, macrophage-mediated, or T-cell-mediated, especially Th1-cell-mediated, immunological disease as well as non-T-cell-mediated pathophysiological inflammation reactions.

As indicated above, T-lymphocytes can be divided according to the expression of their surface antigens into CD4-positive T cells (helper-T cells) and CD8-positive T cells (cytotoxic T cells), and the CD4-helper-T cells (Th cells) in turn into T-helper cells 1 (Th1) and T-helper cells 2 (Th2), which are distinguished, i.a., in their ability to prevent tolerance. The cytokines IL 12 and IFNγ play an important role in the induction and maintaining of the TH1 cell differentiation. Differentiated Th1 cells secrete IFNγ, interleukin 2 and TNFα/β. These cytokines activate in turn macrophages and cytotoxic CD 8 positive T cells.

The invention relates in particular to the use of microglia inhibitors for the production of a pharmaceutical agent for interrupting the IL 12 or IFNγ production in cells of monocytic origin or T cells and NK cells. Based on their ability to interrupt the production of IL 12 and TNFα in monocytes/macrophages/dendrites and the IFNγ production in T cells and NK cells and to increase the induction of the IL-10 production, microglia inhibitors are suitable for treating numerous diseases that are triggered by the increased production of cytokines, such as, e.g., TNFα, β, IFNγ, IL-2 and IL 12, such as inflammatory diseases that are not based on neuroinflammation, autoimmune diseases, allergic and infectious diseases, toxin-induced inflammations, pharmacologically triggered inflammation reactions as well as pathophysiologically relevant inflammation reactions of an origin that is unclear at this time.

Examples of inflammatory and autoimmune diseases are: chronic inflammatory intestinal diseases (inflammatory bowel diseases, Crohn's disease, ulcerative colitis), arthritis, allergic contact dermatitis, psoriasis, pemphigus, asthma, multiple sclerosis, diabetes, type-1 insulin-dependent diabetes mellitus, rheumatoid arthritis, lupus diseases and other collagenoses, Graves' disease, Hashimoto's disease, “graft-versus-host disease” and transplant rejection.

Examples of allergic, infectious and toxin-triggered and ischemia-triggered diseases are: sarcoidosis, asthma, hypersensitive pneumonitis, sepsis, septic shock, endotoxin shock, toxic shock syndrome, toxic liver failure, ARDS (acute respiratory distress syndrome), eclampsia, cachexia, acute virus infections (e.g., mononucleosis, fulminating hepatitis), and post-reperfusion organ damage.

An example of a pharmacologically triggered inflammation with pathophysiological relevance is the “first dose response” after the administration of anti-T-cell antibodies such as OKT3.

An example of systemic inflammation reactions of an origin that is unclear at this time is eclampsia.

Compounds that with stimulation with the Aβ-peptide achieve an inhibition of the microglia activity of at least 20% and an inhibition of the cytokine activity of at least 30% are suitable, according to the invention, as microglia inhibitors. The biological properties of the microglia inhibitors can be shown according to the methods that are known to one skilled in the art, for example with the aid of the testing methods that are described below and in WO 01/51473.

Especially suitable microglia inhibitors with the above-described properties are benzimidazoles of formula I, their tautomeric and isomeric forms and salts.

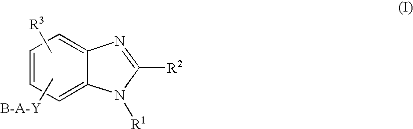

Here:

R¹ is an aryl group or a five- or six-membered heteroaryl group with one or two heteroatoms, selected from the group that comprises N, S and O, whereby the aryl group or heteroaryl group can be substituted with up to three radicals, independently of one another, selected from the group that comprises:

F, Cl, Br,

C(NH)NH₂, C(NH)NHR⁴, C(NH)NR⁴R^{4′}, C(NR⁴)NH₂, C(NR⁴)NHR^{4′},

C(NR⁴)NR⁴R^{4′},

X—OH, X—OR⁴, X—OCOR⁴, X—OCONHR⁴,

X—COR⁴, X—C(NOH)R⁴,

X—CN, X—COOH, X—COOR⁴, X—CONH₂, X—CONR⁴R^{4′}, X—CONHR⁴,

X—CONHOH,

X—SR⁴, X—SOR⁴, X—SO₂R⁴,

SO₂NH₂, SO₂NHR⁴, SO₂NR⁴R^{4′},

NO₂, X—NH₂, X—NHR⁴, X—NR⁴R^{4′}, X—NHSO₂R⁴, X—NR⁴SO₂R^{4′},

X—NHCOR⁴, X—NHCOOR⁴, X—NHCONHR⁴ and

R⁴,

whereby X is a bond, CH₂, (CH₂)₂ or CH(CH₃)₂,

whereby also radicals R⁴ and R^{4′} according to the meanings that are further indicated below are selected independently of one another, and

whereby two substituents at R¹, if they are in ortho-position to one another, can be linked to one another in each case such that together they form a methanediylbisoxy, ethane-1,2-diylbisoxy, propane-1,3-diyl or butane-1,4-diyl group, or

a radical that is selected from the group that comprises C₁₋₆ alyl, (C₀₋₃-alkanediyl-C₃₋₇-cycloalkyl) and C₃₋₆alkenyl,

in which an H atom can be exchanged for a heterocyclic radical that is selected from the group that comprises piperazine, morpholine, piperidine and pyrrolidine, in such a way that a bond to a first N atom of the heterocyclic radical is formed,

whereby the above-mentioned alkyl, cycloalkyl, and alkenyl radicals and the heterocyclic radical can be substituted with up to two radicals that are selected from the group that comprises C₀₋₂-alkanediyl-OH, C₀₋₂-alkanediyl-OR⁷,

C₀₋₂-alkandediyl-NH₂, C₀₋₂-alkanediyl-NHR⁷, C₀₋₂-alkanediyl-NR⁷R^{7′}, C₀₋₂-alkanediyl-NHCOR⁷, C₀₋₂-alkanediyl-NR⁷ COR^{7′}, C₀₋₂-alkanediyl-NHSO₂R⁷, C₀₋₂-alkanediyl-NR⁷SO₂R^{7′}, C₀₋₂-alkanediyl-CO₂H, C₀₋₂-alkanediyl-CO₂R⁷,

C₀₋₂-alkanediyl-CONH₂, C₀₋₂-alkanediyl-CONHR⁷, C₀₋₂-alkanediyl-CONR⁷R^{7′}, phenyl and a five- or six-membered heteroaryl radical,

whereby the heteroaryl radical contains one or two heteroatoms, selected from the group that comprises N, S and O, whereby also the phenyl radical and the heteroaryl radical can be substituted with up to two radicals, selected from the group that comprises F, Cl, Br, CH₃, C₂H₅, OH, OCH₃, OC₂H₅, NO₂, N(CH₃)₂, CF₃, C₂F₅, and SO₂NH₂ and/or also can carry an anellated methanediylbisoxy group or an ethane-1,2-diylbisoxy group,

whereby the piperazine radical on a second nitrogen atom can also be substituted with R⁷, COR⁷ or SO₂R⁷,

whereby R⁷ and R^{7′}, independently of one another, can be selected according to the meanings that are further indicated below,

R is -Z-R^{2′}, an aryl group or a five- or six-membered heteroaryl group with one or two heteroatoms, selected from the group that comprises N, S and O, a benzothienyl group or an indolyl group, whereby the above-mentioned aryl or heteroaryl group can be substituted with up to three radicals, independently of one another, selected from the group that comprises

F, Cl, Br,

C(NH)NH₂, C(NH)NHR⁴, C(NH)NR⁴R^{4′}, C(NR⁴)NH₂, C(NR⁴)NHR^{4′},

C(NR⁴)NR⁴R^{4′},

X—OH, X—OR⁴, X—OCOR⁴, X—OCONHR⁴,

X—COR⁴, X—C(NOH)R⁴,

X—CN, X—COOH, X—COOR⁴, X—CONH₂, X—CONR⁴R^{4′}, X—CONHR⁴,

X—CONHOH,

X—SR⁴, X—SOR⁴, X—SO₂R⁴,

SO₂NH₂, SO₂NHR⁴, SO₂NR⁴R^{4′},

NO₂, X—NH₂, X—NHR⁴, X—NR⁴R^{4′}, X—NHSO₂R⁴, X—NR⁴SO₂R^{4′},

X—NHCOR⁴, X—NHCOOR⁴, X—NHCONHR⁴ and

R⁴,

whereby X is a bond, CH₂, (CH₂)₂, or CH(CH₃)₂,

whereby also radicals R⁴ and R^{4′} are selected independently of one another according to the meanings that are further indicated below, and

whereby two radicals at R¹, if they are in ortho-position to one another, can be linked to one another such that together they form a methanediylbisoxy, ethane-1,2-diylbisoxy-, propane-1,3-diyl or butane-1,4-diyl group,

Z is NH, NR^{2″}, O, S, SO or SO₂, whereby R″ has the meaning that is indicated below,

R^{2′} and R^{2″}, independently of one another, in each case are a radical that is selected from the group that comprises:

C₁₋₄-perfluoroalkyl, C₁₋₅-alkyl, (C₀₋₃-alkanediyl-C₃₋₇ cycloalkyl),

(C₀₋₃-alkanediyl-aryl) and (C₀₋₃-alkanediyl-heteroaryl),

whereby the heteroaryl group is five- or six-membered and contains one or two heteroatoms, selected from the group that comprises N, S and O, and

whereby the aryl group and the heteroaryl group can be substituted in each case with up to two radicals, selected from the group that comprises F, Cl, Br, CH₃, C₂H₅, OH, OCH₃, OC₂H₅, NO₂, N(CH₃)₂, CF₃, C₂F₅and SO₂NH₂ and/or also can carry an anellated methanediylbisoxy group or an ethane-1,2-diylbisoxy group, and

in addition in a five-membered cycloalkyl ring, a ring member can be ring N or ring O, and in a six- or seven-membered cycloalkyl ring, one or two ring members can be ring-N atoms and/or ring-O atoms, whereby the ring-N atoms optionally can be substituted with C₁₋₃-alkyl or C₁₋₃-alkanoyl, alkanoyl,

or R^{2′} and R^{2″} together with Z form a five- to seven-membered heterocyclic ring, if Z is an N atom, whereby Z has the meaning that is further indicated above, whereby also the heterocyclic ring contains another N, O or S atom and optionally can be substituted with a radical that is selected from the group that comprises C₁₋₄-alkyl, (C₀₋₃-alkanediyl-C₁₋₃-alkoxy), C₁₋₄-alkanoyl, C₁₋₄-alkoxycarbonyl, aminocarbonyl and aryl,

R³, independently of one another, are one or two radicals that are selected from the group that comprises:

hydrogen,

F, Cl, Br,

OH, OR⁴, OCOR⁴, OCONHR⁴,

COR⁴,

CN, COOH, COOR⁴, CONH₂, CONHR⁴, CONR⁴R^{4′}, CONHOH,

CONHOR⁴,

SR⁴, SOR⁴, SO₂R⁴, SO₂NH₂, SO₂NHR⁴, SO₂NR⁴R^{4′},

NO₂, NH₂, NHR⁴, NR⁴R^{4′},

NHSO₂R⁴, NR⁴SO₂R^{4′}, NHSO₂R⁶, NR⁴SO₂R⁶,

NHCOR⁴, NHCOOR⁴, NHCONHR⁴ and R⁴,

whereby radicals R⁴, R^{4′} and R⁶ are selected independently of one another according to the meanings that are further indicated below,

A is a group that is selected from the group that comprises C₁₋₁₀-alkanediyl, C₂₋₁₀-alkenediyl, C₂₋₁₀-alkinediyl and (C₀₋₃-alkanediyl-C₃₋₇-cycloalkanediyl-C₀₋₃-alkanediyl),

whereby in a five-membered cycloalkyl ring, a ring member can be ring N or ring O, and in a six- or seven-membered cycloalkyl ring, one or two ring members in each case can be ring-N atoms and/or ring-O atoms,

whereby the ring-N atoms optionally can be substituted with C₁₋₃-alkyl or C₁₋₃-alkanoyl,

whereby in the above-mentioned aliphatic chains, a C atom can be exchanged for O, NH, N—C₁₋₃-alkyl or N—C₁₋₃-alkanoyl and whereby alkyl or cycloalkyl groups optionally can be substituted with a radical that is selected from the group that comprises ═O, OH, O—C₁₋₃-alkyl, NH₂, NH—C₁₋₃-alkyl, NH—C₁₋₃-alkanoyl, N(C₁₋₃-alkyl)₂ and N(C₁₋₃-alkyl)(C₁₋₃-alkanoyl),

B is a radical that is selected from the group that comprises COOH, COOR⁵, CONH₂, CONHNH₂, CONHR⁵, CONR⁵R^{5′}, CONHOH, CONHOR⁵ and tetrazolyl,

in each case bonded to a C atom of group A,

whereby radicals R⁵ and R^{5′}, independently of one another, are selected according to the meanings that are further indicated below,

Y is a group that is selected from the group that comprises O, NH, NR⁴, NCOR⁴, NSO₂R⁴ and NSO₂R⁶,

whereby R⁴ and R⁶ have the meanings that are further indicated below, in which in the radicals above, radicals R⁴, R^{4′}, R⁵, R^{5′} and R⁶ have the following meanings; here:

R⁴ and R^{4′}, independently of one another, in each case are a radical that is selected from the group that comprises CF₃, C₂F₅, C₁₋₄-alkyl, C₂₋₄-alkenyl, C₂₋₃-alkinyl and

(C₀₋₃-alkanediyl-C₃₋₇-cycloalkyl),

whereby in a five-membered cycloalkyl ring, a ring member can be ring N or ring O, and in a six- or seven-membered cycloalkyl ring, one or two ring members in each case can be ring-N atoms and/or ring-O atoms,

whereby the ring-N atoms optionally can be substituted with C₁₋₃-alkyl or C₁₋₃-alkanoyl,

R⁵ and R^{5′}, independently of one another, are in each case a radical that is selected from the group that comprises C₁₋₆-alkyl, C₂₋₆-alkenyl, C₂₋₆-alkinyl, whereby a C atom can be exchanged for O, S, SO, SO₂, NH, N—C₁₋₃-alkyl or N—C₁₋₃-alkanoyl, and also (C₀₋₃-alkanediyl-C₃₋₇-cycloalkyl), whereby in a five-membered cycloalkyl ring, a ring member can be ring N or ring O, and in a six- or seven-membered cycloalkyl ring, one or two ring members in each case can be ring-N atoms and/or ring-O atoms, whereby the ring-N atoms optionally can be substituted with C₁₋₃-alkyl or C₁₋₃-alkanoyl, as well as also (C₀₋₃-alkanediyl-aryl) and (C₀₋₃-alkanediyl-heteroaryl), whereby the heteroaryl group is five- or six-membered and contains one or two heteroatoms that are selected from the group that comprises N, S and O,

whereby all above-mentioned alkyl and cycloalkyl radicals can be substituted with up to two radicals that are selected from the group that comprises CF₃, C₂F₅, OH, O—C₁₋₃-alkyl, NH₂, NH—C₁₋₃-alkyl, NH—C₁₋₃-alkanoyl, N(C₁₋₃-alkyl)₂, N(C₁₋₃-alkyl)(C₁₋₃-alkanoyl), COOH, CONH₂ and COO—C₁₋₃-alkyl, and all above-mentioned aryl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂ and/or also can carry an anellated methanediylbisoxy group or ethane-1,2-diylbisoxy group,

or R⁵ and R^{5′} together with the amide-N atom of B form a five- to seven-membered, saturated or unsaturated heterocyclic ring that can contain another N or O or S atom and that can be substituted with C₁₋₄-alkyl, (C₀₋₂-alkanediyl-C₁₋₄-alkoxy), C₁₋₄-alkoxycarbonyl, aminocarbonyl or aryl,

R⁶ is a radical that is selected from the group that comprises (C₀₋₃-alkanediyl-aryl) and (C₀₋₃-alkanediyl-heteroaryl), whereby the heteroaryl group is five- or six-membered and contains one or two heteroatoms that are selected from the group that comprises N, S and O, and whereby the aryl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂, and/or also can carry an anellated methanediylbisoxy group or an ethane-1,2-diylbisoxy group,

R⁷ and R^{7′}, independently of one another, are R⁴ or R⁶.

Preferred are those benzimidazole derivatives in which substituent B-A-Y is bonded to the 6-position of the benzimidazole.

This invention also comprises physiologically compatible salts as well as esters of the above-mentioned compounds, especially the acid salts of the nitrogen bases of the benzimidazole derivatives according to the invention, also the salts of carboxylic acids of the derivatives according to the invention with bases as well as the esters of the carboxylic acids of the derivatives, as well as carboxylic acids that are derived from carboxylic acid derivatives, for example carboxylic acid amides.

The benzimidazole derivatives can have a chiral center or several chiral centers, such that the compounds can occur in several isomeric forms. The compounds of formula I can also be present as tautomers, stereoisomers or geometric isomers. The invention also comprises all possible isomers, such as E- and Z-isomers, S- and R-enantiomers, diastereomers, racemates and mixtures thereof including the tautomeric compounds. All of these isomeric compounds are—even if not expressly indicated otherwise in each case—components of this invention. The isomer mixtures can be separated into enantiomers or E/Z-isomers according to commonly used methods, such as, for example crystallization, chromatography or salt formation.

The heteroaryl groups that are contained in the benzimidazole compounds are built up from five or six skeleton atoms and can contain one or two heteroatoms. Heteroatoms are oxygen (O), nitrogen (N) and sulfur (S). Examples of heteroaryl groups are pyrrolyl, thienyl, furanyl, imidazolyl, thiazolyl, isothiazolyl, oxazolyl, isoxazolyl, pyrazolyl, pyridyl, pyrimidinyl, pyrazinyl and pyridazinyl. If the heteroaryl groups are part of R¹ or R², the group is bonded via a C atom to the respective N atom of the benzimidazole skeleton or to substituent Z.

As aryl radicals, primarily the phenyl radicals, but also the naphthyl radicals are suitable. The aryl and heteroaryl radicals can be bonded in any way to the benzimidazole skeleton or another group, for example as a 1- or 2-naphthyl or as a 2-, 3- or 4-pyridinyl.

Alkyl groups can be straight-chain or branched. Examples of alkyl groups are methyl, ethyl, n-propyl, iso-propyl, n-butyl, sec-butyl, tert-butyl, n-pentyl, sec-pentyl, tert-pentyl, neo-pentyl, n-hexyl, sec-hexyl, heptyl, octyl, nonyl, and decyl. The higher homologs in each case also comprise both the linear alkyl groups and the branched alkyl groups, thus, for example, 2-ethylhexyl for octyl and 3-propyl-hexyl for nonyl.

Perfluorinated alkyls are preferably CF₃ and C₂F₅.

Alkenyl groups can be straight-chain or branched. For example, vinyl, 2-propenyl, 1-propenyl, 2-butenyl, 1-butenyl, 1-methyl-1-propenyl, 2-methyl-2-propenyl and 3-methyl-2-propenyl are alkenyl radicals in terms of the invention.

Alkinyl groups can be straight-chain or branched. Examples of this are ethinyl, 1-propinyl, 2-propinyl, 1-butinyl and 2-butinyl.

Cycloalkyl groups are preferably defined in each case as cyclopropyl, cyclobutyl, cyclopentyl, cyclohexyl or cycloheptyl (corresponds to C₃₋₇-cycloalkyl).

Mentioned as a saturated heterocyclic ring or as a cycloalkyl with one or more heteroatoms are, for example: piperidine, pyrrolidine, tetrahydrofuran, morpholine, piperazine, hexahydroazepine as well as 2,6-dimethyl-morpholine, N-phenyl-piperazine, and methoxymethyl-pyrrolidine, whereby the linkage with a C atom that is adjacent to the ring can be carried out via optionally present ring-N atoms.

Alkanediyl, alkenediyl, alkinediyl and cycloalkanediyl radicals that are mentioned in the description of the invention are the same in meaning as alkylene, alkenylene, alkinylene and cycloalkylene. If the number of the C atoms contained is indicated in the general formulas of the alkanediyl radicals and the value of 0 is indicated as a lower range limit of this number, this alkanediyl radical is not contained in the respective case.

Mentioned as alkanes, alkenes, and alkines for A are, for example: straight-chain or branched alkanediyl with one to eight C atoms, for example methanediyl, ethanediyl, propanediyl, butanediyl, pentanediyl, hexanediyl, also 1-methylethanediyl, 1-ethylethanedlyl, 1-methylpropanediyl, 2-methylpropanediyl, 1-methylbutanediyl, 2-methylbutanediyl, 1-ethylbutanediyl, 2-ethylbutanediyl, 1-methylpentanediyl, 2-methylpentanediyl, and 3-methyl-pentanediyl as well as analogous compounds.

Straight-chain or branched alkenediyl and alkinediyl with two to eight C atoms are alkenediyl groups or alkinediyl groups with double and triple bonds in all possible positions as well as with all possible methyl or ethyl substitutions. In these radicals, one or two C atoms can be exchanged for O, NH, N—C₁₋₃-alkyl or N—C₁₋₃-alkanoyl in each case, whereby the exchanged group is separated from Y by at least two C atoms.

If two radicals are in ortho-position, they can form a common ring with the adjacent aromatic compound. Compounds in which N, O or S atoms are bonded to olefinic or acetylenic multiple bonds, or in which several N, O, S or halogen atoms are bonded to the same aliphatic C atom, or in which N, O or S atoms are bonded directly to one another, are excluded if these linkages are not defined explicitly, for example in the functional groups or in heteroaromatic compounds that are mentioned in the claim.

The physiologically compatible acid salts of the nitrogen bases of the benzimidazole derivatives according to the invention can be formed with inorganic and organic acids, for example with oxalic acid, lactic acid, citric acid, fumaric acid, acetic acid, maleic acid, tartaric acid, phosphoric acid, hydrochloric acid, hydrobromic acid, sulfuric acid, p-toluenesulfonic acid and methanesulfonic acid.

For salt formation of acid groups, especially carboxylic acid groups, the inorganic or organic bases that are known for forming physiologically compatible salts, such as, for example, alkali hydroxides, especially sodium hydroxide and potassium hydroxide, alkaline-earth hydroxides, such as calcium hydroxide, also ammonia, as well as amines, such as ethanolamine, diethanolamine, triethanolamine, N-methylglucamine and tris-(hydroxymethyl)-methylamine, are also suitable.

For ester formation, all lower monovalent, divalent and trivalent alcohols are suitable, especially methanol, ethanol, iso-propanol and tert-butanol as well as ethylene glycol and glycerol.

Especially preferred are benzimidazoles with the general formula I, in which the radicals and groups that are indicated below independently of one another have the following meanings:

R¹ means a phenyl group, which can be substituted with up to two radicals, independently of one another, that are selected from the group that comprises:

F, Cl, Br,

C(NH)NH₂, C(NH)NHR⁴, C(NH)NR⁴R^{4′}, C(NR⁴)NH₂, C(NR⁴)NHR^{4′},

C(NR⁴)NR⁴R^{4′},

OH, OR⁴, OCOR⁴, OCONHR⁴,

COR⁴, C(NOH)R⁴,

CN, COOH, COOR⁴, CONH₂, CONR⁴R^{4′}, CONHR⁴, CONHOH,

SR⁴, SOR⁴, SO₂R⁴,

SO₂NH₂, SO₂NHR⁴, SO₂NHR⁴R^{4′},

NO₂, NH₂, NHR⁴, NR⁴R^{4′}, NHCONHR⁴ and

R⁴,

whereby radicals R⁴ and R^{4′} are selected independently of one another according to meanings that are indicated below and whereby two substituents at R¹ can be linked to one another such that together they form a methanediylbisoxy group, ethane-1,2-diylbisoxy group, propane-1,3-diyl group or butane-1,4-diyl group, if they are in ortho-position to one another,

R² means a monocyclic or bicyclic C₆₋₁₀-aryl group or a monocyclic or bicyclic 5- to 10-membered heteroaryl group with 1-2 heteroatoms that are selected from the group that consists of N, S or O, whereby the above-mentioned aryl group or heteroaryl group can be substituted with up to three of the following substituents, independently of one another:

F, Cl, Br,

XOH, XOR⁴, XOCOR⁴, XOCONHR⁴, XOCOOR⁴,

XCOR⁴, XC(NOH)R⁴, XC(NOR⁴)R⁴, XC(NO(COR⁴))R⁴,

XCOOH, XCOOR⁴, XCONH₂, XCONHR⁴, XCONR⁴R⁴, XCONHOH,

XCONHOR⁴, XCOSR⁴,

XSR⁴, XSOR⁴, XSO₂R⁴, SO₂NH₂, SO₂NHR⁴, SO₂NR⁴R⁴,

NO₂, XNHR⁴, XNR⁴R⁴, XNHSO₂R⁴, XN(SO₂R⁴) SO₂R⁴, XNR⁴SO₂R⁴ and

R⁴,

whereby two substituents at R², if they are in ortho-position to one another, can be linked to one another such that together they form methanediylbisoxy, ethane-1,2-diylbisoxy, propane-1,3-diyl, or butane-1,4-diyl,

R³ means a radical that is selected from the group that comprises hydrogen, F, Cl, Br, CH₃, C₂H₅, CF₃, C₂F₅, OH, OR⁴, NHSO₂R⁶ and NHCOR⁴,

whereby R⁴ and R⁶ have the meanings that are further indicated below,

A means C₁₋₁₀-alkanediyl, C₂₋₁₀-alkenediyl, C₂₋₁₀-alkinediyl, (C₀₋₅-alkanediyl-C₃₋₇-cycloalkanediyl-C₀₋₅-alkanediyl),

whereby in a 5-membered cycloalkyl ring, a ring member can be an N or an O, and in a 6- or 7-membered cycloalkyl ring, one or two ring members can be N and/or O, whereby ring nitrogens optionally can be substituted with C₁₋₃-alkyl or C₁₋₃-alkanoyl,

whereby in the above-mentioned aliphatic chains, a carbon atom or two carbon atoms can be exchanged for O, NH, NC₁₋₃-alkyl, NC₁₋₃-alkanoyl,

B means a radical that is selected from the group that comprises COOH, COOR⁵, CONH₂, CONHR⁵ and CONR⁵R^{5′}, in each case bonded to a C atom of group A,

whereby radicals R⁵ and R^{5′} can be selected independently of one another according to the meanings that are further indicated below,

Y means O,

in which in the above radicals, radicals R⁴, R^{4′}, R⁵, R^{5′} and R⁶ have the following meanings; here:

R⁴ and R^{4′} have the same meaning as further indicated above,

R⁵ and R^{5′}, independently of one another, in each case mean a radical that is selected from the group that comprises C₁₋₆-alkyl, C₂₋₆-alkenyl, C₂₋₆-alkinyl, whereby a C atom can be exchanged for O, S, SO, SO₂, NH, N—C₁₋₃-alkyl or N—C₁₋₃-alkanoyl, also (C₀₋₃-alkanediyl-C₃₋₇-cycloalkyl),

whereby in a five-membered cycloalkyl ring, a ring member can be ring N or ring O, and in a six- or seven-membered cycloalkyl ring, one or two ring members in each case can be ring-N atoms and/or ring-O atoms,

whereby the ring-N atoms optionally can be substituted with C₁₋₃-alkyl or C₁₋₃-alkanoyl, as well as also (C₀₋₃-alkanediyl-phenyl) and (C₀₋₃-alkanediyl-heteroaryl), whereby the heteroaryl group is five- or six-membered and contains one or two heteroatoms that are selected from the group that comprises N, S and O,

whereby all above-mentioned alkyl and cycloalkyl radicals can be substituted with a radical that is selected from the group that comprises CF₃, C₂F₅, OH, O—C₁₋₃-alkyl, NH₂, NH—C₁₋₃-alkyl, NH—C₁₋₃-alkanoyl, N(C₁₋₃-alkyl)₂, N(C₁₋₃-alkyl)(C₁₋₃-alkanoyl), COOH, CONH₂ and COO—C₁₋₃-alkyl, and all above-mentioned phenyl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂ and/or also can carry an anellated methanediylbisoxy group or ethane-1,2-diylbisoxy group,

or R⁵ and R^{5′} together with the amide-N atom of B form a five- to seven-membered, saturated or unsaturated heterocyclic ring that can contain another N or O or S atom and that can be substituted with C₁₋₄-alkyl, (C₀₋₂-alkanediyl-C₁₋₄-alkoxy), C₁₋₄-alkoxycarbonyl, aminocarbonyl or phenyl,

R⁶ means a phenyl or heteroaryl group, whereby the heteroaryl group is five- or six-membered and contains one or two heteroatoms that are selected from the group that comprises N, S and O, and whereby the phenyl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂, or else can carry an anellated methanediylbisoxy group or ethane-1,2-diylbisoxy group,

R³ is preferably hydrogen. The grouping Y-A is presented in a preferred embodiment by a C₁₋₆-alkylenoxy group that is bonded to the benzimidazole skeleton via the O atom.

The invention also relates to pharmaceutical agents that contain one or more compounds of general formula I as well as one or more vehicles. The pharmaceutical agents or compositions of the invention are produced in a way that is known in the art with commonly used solid or liquid vehicles or diluents and commonly used pharmaceutical and technical adjuvants that correspond to the desired type of administration with a suitable dosage. Preferred preparations consist in a dispensing form that is suitable for oral, enteral or parenteral administration, for example i.p. (intraperitoneal), i.v. (intravenous), i.m. (intramuscular) or percutaneous administration. Such dispensing forms are, for example, tablets, film tablets, coated tablets, pills, capsules, powders, creams, ointments, lotions, liquids, such as syrups, gels, injectable liquids, for example for i.p., i.v., i.m. or percutaneous injection, etc. In addition, depot forms such as implantable preparations, as well as suppositories, are also suitable. In this case, depending on their type, the individual preparations release to the body the derivatives according to the invention gradually or all at once in a short time.

For oral administration, capsules, pills, tablets, coated tablets and liquids or other known oral forms for dispensing can be used as pharmaceutical preparations. In this case, the pharmaceutical agents can be formulated in the way that they release the active ingredients either in a short time and pass on to the body or have a depot action, so that a longer-lasting, slow supply of active ingredients to the body is achieved. In addition to at least one benzimidazole derivative, the dosage units can contain one or more pharmaceutically compatible vehicles, for example substances for adjusting the rheology of the pharmaceutical agent, surfactants, solubilizers, microcapsules, microparticles, granulates, diluents, binders, such as starches, sugar, sorbitol and gelatins, also fillers, such as silicic acid and talc, lubricants, dyes, perfumes and other substances.

Corresponding tablets can be obtained by, for example, mixing an active ingredient with known adjuvants, for example inert diluents such as dextrose, sugar, sorbitol, mannitol, polyvinyl pyrrolidone, explosives such as corn starch or alginic acid, binders such as starch or gelatin, lubricants such as carboxypolymethylene, carboxy methyl cellulose, cellulose acetate phthalate or polyvinyl acetate. The tablets can also consist of several layers.

Coated tablets can be produced accordingly by coating cores that are produced analogously to the tablets with agents that are commonly used in tablet coatings, for example polyvinyl pyrrolidone or shellac, gum arabic, talc, titanium oxide or sugar. In this case, the shell of the coated tablet can also consist of several layers, whereby the adjuvants that are mentioned above in the case of the tablets can be used.

Capsules that contain active ingredients can be produced, for example, by the active ingredient being mixed with an inert vehicle such as lactose or sorbitol and encapsulated in gelatin capsules.

The active ingredients can also be formulated in the form of a solution that is intended for oral administration and that in addition to the active benzimidazole derivative contains as components a pharmaceutically compatible oil and/or a pharmaceutically compatible lipophilic surfactant and/or a pharmaceutically compatible hydrophilic surfactant and/or a pharmaceutically compatible water-miscible solvent.

To achieve better bio-availability of the active ingredients, the compounds can also be formulated as cyclodextrin clathrates. To this end, the compounds are reacted with α-, β-, or γ-cyclodextrin or derivatives thereof.

If creams, ointments, lotions and liquids that can be applied topically are to be used, the latter must be constituted so that the compounds are fed to the body in adequate amounts. In these forms for dispensing, adjuvants are contained, for example substances for adjusting the rheology of pharmaceutical agents, surfactants, preservatives, solubilizers, diluents, substances for increasing the permeability of the active ingredients through the skin, dyes, perfumes and skin protection agents such as conditioners and moisturizers. Together with the compounds, other active ingredients can also be contained in the pharmaceutical agent [Ullmanns Enzyklopädie der technischen Chemie [Ullmann's Encyclopedia of Technical Chemistry], Volume 4 (1953), pages 1-39; J. Pharm. Sci., 52, 918 ff. (1963); issued by Czetsch-Lindenwald, Hilfsstoffe für Pharmazie und angrenzende Gebiete [Adjuvants for Pharmaceutics and Related Fields]; Pharm. Ind., 2, 72 ff (1961); Dr. H. P. Fiedler, Lexikon der Hilfsstoffe für Pharmazie, Kosmetik und angrenzende Gebiete [Dictionary of Adjuvants for Pharmaceutics, Cosmetics and Related Fields], Cantor, A. G., Aulendorf/Württ., 1971].

The substances can also be used in suitable solutions such as, for example, physiological common salt solution, as infusion or injection solutions. For parenteral administration, the active ingredients can be dissolved or suspended in a physiologically compatible diluent. As diluents, in particular oily solutions, such as, for example, solutions in sesame oil, castor oil and cottonseed oil, are suitable. To increase solubility, solubilizers, such as, for example, benzyl benzoate or benzyl alcohol, can be added.

To formulate an injectable preparation, any liquid vehicle can be used in which the compounds according to the invention are dissolved or emulsified. These liquids frequently also contain substances to regulate viscosity, surfactants, preservatives, solubilizers, diluents and other additives, with which the solution is set to isotonic. Other active ingredients can also be administered together with the benzimidazole derivatives.

It is also possible to incorporate the substances in a transdermal system and thus to administer them transdermally. To this end, the benzimidazole derivatives are applied in the form of a depot injection or an implant preparation, for example subcutaneously. Such preparations can be formulated in such a way that a delayed release of active ingredients is made possible. To this end, known techniques can be used, for example depots that dissolve or operate with a membrane. Implants can contain as inert materials, for example, biodegradable polymers or synthetic silicones, for example silicone gum. The benzimidazole derivatives can also be incorporated in, for example, a patch, for percutaneous administration.

The dosage of the substances is determined by the attending physician and depends on, i.a., the substance that is administered, the method of administration, the disease that is to be treated and the severity of the disease. The daily dose is no more than 1000 mg, preferably no more than 100 mg, whereby the dose can be given as a single dose to be administered once or divided into two or more daily doses.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

Study of the Biological Properties

### EXAMPLE 1

Activation of Macrophages

To test substances on macrophages/monocytes, LPS-activated THP-1 cells were used. For this purpose, 2.5×106 cells/ml were grown in RPMI medium (RPMI 1640+10% FCS). The compounds according to the invention were added at a concentration of 5 μM and pre-incubated for 30 minutes. The stimulation of the cells was carried out overnight at 37° C. with 1 μg/ml of LPS. Then, the medium was harvested, and the TNFα amount was determined quantitatively. The treatment of the cells with the substances according to the invention resulted in a reduction of the TNFα amount of at least 30%.

### EXAMPLE 2

Inhibition of the TNFα- and IL 12-Production in THP-1 Cells

The inhibition of the cytokine production can be visualized, for example, by measuring TNFα and interleukin 12 in lipopolysaccharide (LPS)-stimulated THP-1 cells.

For this purpose, 2.5×10⁵ THP-1 cells (American Type Culture Company, Rockville, Md.)/ml of RPMI 1640 medium (Life Technologies)/10% FCS (Life Technologies, Cat. No. 10270-106) are grown on 96-hole flat-bottomed cell culture plates (TPP, Product No. 9296) (100 μl/hole). The compounds according to the invention are added at various concentrations and pre-incubated for 30 minutes. The pre-dilution of the test substances was performed in the incubation medium. The addition of the test substances is carried out as a 2× concentrated substance solution (100 μl/hole). The stimulation of the cells was carried out overnight at 37° C. with 0.1 μg/ml of LPS (Sigma L2630, of E. Coli Serotype 0111.B4). Then, the medium was harvested, and the amount of TNFα or the amount of interleukin 12 was quantitatively determined. To measure the TNFα, a commercially available TNFα kit of the CIS Bio International Company was used (Product No. 62TNFPEB). The amount of interleukin 12 was implemented with the aid of the ORIGEN technology (IGEN International, Inc., Gaithersburg, Md.). The calculated IC50 value corresponds to the concentration of test substance that is required to reach a 50% inhibition of the maximum TNFα or interleukin 12 production.

In this test batch, the substances show an IC50 of below 10 μM. With the aid of similar methods, the inhibition of IL12 and TNFα with the substances also can be produced with the aid of peripheral blood leukocytes and comparable stimuli.

### EXAMPLE 3

Inhibition of the IFNγ Production of Peripheral Mononuclear Blood Cells

To visualize the effect of the substances on T-cell activation, for example, the measurement of the IFNγ-secretion can be used.

To isolate peripheral mononuclear cells, human whole blood was used (drawing of blood via Na-citrate S-monovettes “Coagulation 9 NC/10 ml”/Sarstedt). The build-up of the blood cells was accomplished with the aid of density gradient centrifuging: For this purpose, 15 ml of Histopaque 1077 (Sigma, Cat. No. H8880) in LEUCOSEP tubes (Greiner, Cat. No. 227290) is introduced and centrifuged for 30 seconds at 1000 g. Then, 15 ml of whole blood is added and centrifuged for 10 minutes at 1000 g. Finally, the upper plasma layer is pipetted off and the subjacent cell layer (peripheral mononuclear blood cells) is transferred in 15 ml sample tubes (Falcon) and then grown several times with 10 ml of HBSS (HANKS Balanced Solution (without Mg2+ and Ca2+), Cat. No. 14175-53). Finally, the cell pellet is resuspended in culture medium RPMI 1640+25 mmol of Hepes (Life Technologies Cat. No. 52400-041 10% FCS (Life Technologies, Cat. No. 10270-106), 0.4% penicillin-streptomycin solution (Life Technologies, Cat. No. 1514-106) (1×10⁶ cells/ml). In each case, 100 μl of cell suspension solution was dispersed on 96-hole flat-bottomed cell culture plates (TPP, Product No. 9296) and stimulated with 5 μg/ml of phytohemagglutinin. The substances according to the invention were added at various concentrations and pre-incubated for 30 minutes. The stimulation of the cells was carried out over a period of 48 hours.

Then the medium was harvested, and the amount of IFNγ was determined quantitatively. The amount of IFNγ was determined with the aid of the ORIGEN technology (IGEN International, Inc., Gaithersburg, Md.). The calculated IC50 value corresponds to the concentration of test substance that is required to reach a 50% inhibition of the maximum TFNγ production.

The treatment of the cells with the test substances resulted in a reduction of the amount of IFNγ by at least 30% at a concentration of 10 μM. With the aid of a similar method, the inhibition of IFNγ with the substances can also be produced with the aid of specific T-cell activators, such as, e.g., monoclonal anti-CD3 antibodies.

TABLE 1

| | Inhibition of | Inhibition of | Inhibition of | Inhibition of |
|---|---|---|---|---|
| | the IL 12 | the TNFα | the IFNγ | the Microglia |
| | Secretion | Secretion | Secretion | Activation |
| Substance | (IC₅₀) | (IC₅₀) | (IC₅₀) | (IC₅₀) |
| 1 | 2.2 μM | 3 μM | 1.3 μM | 0.75 μM |
| 2 | <0.3 μM | 0.15 μM | 0.11 μM | 0.16 μM |

Substance 1:
6-[[1-(4-Methylphenyl)-2-phenyl-1H-benzimidazol-6-yl]oxy]hexanoic acid-isopropyl ester
Substance 2:
6-[[1-(4-Methylphenyl)-2-phenyl-1H-benzimidazol-6-yl]oxy]hexanoic acid

### EXAMPLE 4

Induction of IL-10 Production of Peripheral Mononuclear Blood Cells

The induction of IL-10 production is visualized by, for example, measuring IL-10 in peripheral mononuclear blood cells that are stimulated in phytohemagglutinin (PHA) or lipopolysaccharide (LPS).

To isolate peripheral mononuclear blood cells, human whole blood was used (drawing of blood via Na-Citrate S-monovettes “Coagulation 9 NC/10 ml”/Sarstedt). The build-up of the lymphocytes and monocytes was accomplished with the aid of the density gradient centrifuging: For this purpose, 15 ml of Histopaque-1077 (Sigma, Cat. No. H8880) in 50 ml LEUCOSEP tubes (Greiner, Cat. No. 227290) is introduced and forced downward by centrifuging for 30 seconds at 250 g by the frits that are contained in the tubes. Then, 20 ml of whole blood is added and centrifuged for 15 minutes at 800 g and at room temperature. After centrifuging, the supernatant (plasma and thrombocytes) is pipetted off and discarded, and the subjacent cell layer (lymphocytes and monocytes) in 50 ml centrifuging tubes (Falcon) is transferred and then washed 3× in culture medium VLE RPMI 1640 (Seromed, No. FG1415) (centrifuging in each case for 10 minutes at 250 g, room temperature). Finally, the cell pellet is resuspended in culture medium VLE RPMI 1640 (Seromed, No. FG1415), 10% FCS (Life Technologies, Cat. No. 16000-044, low endotoxin, heat-inactivated for 1 hour, 56° C.), 50 μg/ml of penicillin-streptomycin solution (Life Technologies, Cat. No. 15140-106) and set at 3×10⁶ cells/ml after cell counting by means of trypan-blue staining. In each case, 100 μl of cell suspension solution was dispersed on 96-hole flat-bottomed cell culture plates (Costar, Product No. 3599). In each case, 100 μl of 3×-concentrated stimulation solution (3 μg/ml of LPS of E. coli Serotype 0127:B8; Sigma, Cat. No. L-4516 or 300 μl/ml of PHA-L, Biochrom KG, Cat. No. M5030) was added to it. The substances according to the invention were added at various concentrations as 3×-concentrated substance solution (100 μl/well). The stimulation of the cells was carried out at 37° C. and 5% CO₂ over a period of 24 hours. Then, the cell culture supernatant was harvested, and IL-10 was determined quantitatively. The IL-10 concentration was determined by means of a commercially available ELISA Kit of the BioSource International Company (human IL-10, Cat. No. KHC0101C). The calculated EC₅₀ value corresponds to the concentration of test substance that is required to increase the IL-10 secretion by 50% of the maximum increase.

The compounds according to the invention increase the IL-10 production of peripheral mononuclear blood cells.

### EXAMPLE 5

Inhibition of the TNFα and IL-12 HD Production of Peripheral Mononuclear Blood Cells

The inhibition of the TNFα and IL-12 HD p70 production is visualized by, for example, measuring TNFα and IL-12 HD p70 in peripheral mononuclear blood cells that are stimulated with lipopolysaccharide (LPS) and interferon gamma (IFNγ).

To isolate peripheral mononuclear blood cells, human whole blood was used (drawing of blood via Na-Citrate S-monovettes “Coagulation 9 NC/10 ml”/Sarstedt). The build-up of the lymphocytes and monocytes was accomplished with the aid of the density gradient centrifuging: For this purpose, 15 ml of Histopaque-1077 (Sigma, Cat. No. H8880) is introduced into 50 ml LEUCOSEP tubes (Greiner, Cat. No.227290) and forced downward by centrifuging for 30 seconds at 250 g by the frits that are contained in the tubes. Then, 20 ml of whole blood is added and centrifuged for 15 minutes at 800 g and at room temperature. After centrifuging, the supernatant (plasma and thrombocytes) is pipetted off and discarded, and the subjacent cell layer (lymphocytes and monocytes) is transferred into 50 ml centrifuging tubes (Falcon) and then washed 3× in culture medium VLE RPMI 1640 (Seromed, No. FG1415) (centrifuging in each case for 10 minutes at 250 g, room temperature). Finally, the cell pellet is resuspended in culture medium VLE RPMI 1640 (Seromed, No. FG1415), 10% FCS (Life Technologies, Cat. No. 16000-044, low endotoxin, heat-inactivated for 1 hour, 56° C.), and 50 μg/ml of penicillin-streptomycin solution (Life Technologies, Cat. No. 15140-106) and set at 3×10⁶ cells/ml after cell counting by means of trypan blue staining. In each case, 100 μl of cell suspension solution was dispersed on 96-hole flat-bottomed cell culture plates (Costar, Product No. 3599). In each case, 100 μl of 3×-concentrated stimulation solution (3 μg/ml of LPS of E. coli Serotype 0127:B8; Sigma, Cat. No. L-4516 and 300 ng/ml of IFNγ 1b, Imukin, Boehringer Ingelheim) was added to it. The substances according to the invention were added at various concentrations as 3×-concentrated substance solution (100 μl/well). The stimulation of the cells was carried out at 37° C. and 5% CO₂ over a period of 24 hours. Then, the cell culture supernatant was harvested, and the concentrations of TNFα and IL-12 HD p70 were determined by means of commercially available ELISA Kits of the BioSource International Company (TNF-α EASIA, Cat. No. KAC1752) and R & D Systems (Quantikine™ HS IL-12, Cat. No. HS 120).

The calculated IC₅₀ value corresponds to the concentration of test substance that is required to reach a 50% inhibition of the maximum TNFα or interleukin 12 HD p70 production.

The compounds according to the invention inhibit the TNFα and IL-12 HD p70 production of peripheral mononuclear blood cells.

TABLE 2

| | Inhibition of IL-l2HD | | Inhibition of TNF-alpha | | Induction of the IL-10 | |
|---|---|---|---|---|---|---|
| | Secretion after LPS & | | Secretion after LPS & | | Secretion after PHA | |
| | IFNg Stimulation | | IFNg Stimulation | | Stimulation | |
| | IC50 | Effectiveness | IC50 | Effectiveness | EC50 | Effectiveness |
| Substance | (mol/l) | (%) | (mol/l) | (%) | (mol/l) | |
| 1 | 9.2E−08 | 81 | 6.5E−07 | 65 | >1.0E−6 | 245 |
| 2 | 3.0E−08 | 93 | 1.2E−07 | 83 | 1.8E−07 | 283 |

Substance 1:
6-[[1 -(4-Methylphenyl)-2-phenyl-1H-benzimidazol-6-yl]oxy]hexanoic acid-isopropyl ester
Substance 2:
6-[[1-(4-Methylphenyl)-2-phenyl-1H-benzimidazol-6-yl]oxy]hexanoic acid

The entire disclosures of all applications, patents and publications, cited herein and of corresponding German Application No. 102 07 843.2, filed Feb. 15, 2002, and U.S. Provisional Application Serial No. 60/357,833, filed Feb. 21, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of a microglia inhibitor for the production of a pharmaceutical agent for treating immune reactions that are mediated by monocytes, macrophages or T cells.

2. Use according to claim 1 for treating immune reactions that are mediated by interleukin 12 (IL 12) and interferon γ (IFNγ).

3. Use according to claim 1 for treating non-T-cell-mediated inflammation reactions.

4. Use according to claim 1 for treating autoimmune diseases, inflammatory diseases that are not based on neuroinflammation, allergic and infectious diseases.

5. Use according to claim 4 for treating chronic inflammatory intestinal diseases, for example inflammatory bowel diseases, Crohn's disease, or ulcerative colitis, arthritis, allergic contact dermatitis, psoriasis, pemphigus, asthma, diabetes, type-1 insulin-dependent diabetes mellitus, rheumatoid arthritis, lupus diseases and other collagenoses, Graves' disease, Hashimoto's disease, “graft-versus-host disease” and transplant rejection, sarcoidosis, asthma, hypersensitive pneumonitis, sepsis, septic shock, endotoxin shock, toxic shock syndrome, toxic liver failure, ARDS (acute respiratory distress syndrome), eclampsia, cachexia, acute virus infections, post-reperfusion organ damage, “first-dose response” after administration of anti-T-cell antibodies.

6. Use of a benzimidazole of formula I, its tautomeric or isomeric form or salt 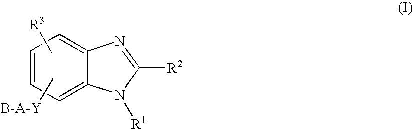
in which
R¹means an aryl group or a five- or six-membered heteroaryl group with one or two heteroatoms, selected from the group that comprises N, S and O, whereby the aryl group or heteroaryl group can be substituted with up to three radicals, independently of one another, selected from the group that comprises:
F, Cl, Br,
C(NH)NH₂, C(NH)NHR⁴, C(NH)NR⁴R^{4′}, C(NR⁴)NH₂, C(⁴)NHR^{4′},
C(NR⁴)NR⁴R^{4′},
X—OH, X—OR⁴, X—OCOR⁴, X—OCONHR⁴,
X—COR⁴, X—C(NOH)R⁴,
X—CN, X—COOH, X—COOR⁴, X—CONH₂, X—CONR⁴R^{4′}, X—CONHR⁴,
X—CONHOH,
X—SR⁴, X—SOR⁴, X—SO₂R⁴,
SO₂NH₂, SO₂NHR⁴, SO₂NR⁴R^{4′},
NO₂, X—NH₂, X—NHR⁴, X—NR⁴R^{4′}, X—NHSO₂R⁴, X—NR⁴SO₂R^{4′},
X—NHCOR⁴, X—NHCOOR⁴, X—NHCONHR⁴ and
R⁴,
whereby X is a bond, CH₂, (CH₂)₂ or CH(CH₃)₂,
whereby also radicals R⁴ and R^{4′} according to the meanings that are further indicated below are selected independently of one another, and whereby two substituents at R¹, if they are in ortho-position to one another, can be linked to one another in each case such that together they form a methanediylbisoxy, ethane-1,2-diylbisoxy, propane-1,3-diyl or butane-1,4-diyl group, or
a radical that is selected from the group that comprises C₁₋₆ alkyl, (C₀₋₃-alkanediyl-C₃₋₇-cycloalkyl) and C₃₋₆alkenyl,
in which an H atom can be exchanged for a heterocyclic radical that is selected from the group that comprises piperazine, morpholine, piperidine and pyrrolidine, such that a bond to a first N atom of the heterocyclic radical is formed,
whereby the above-mentioned alkyl, cycloalkyl, and alkenyl radicals and the heterocyclic radical with up to two radicals can be substituted. selected from the group that comprises C₀₋₂-alkanediyl-OH, C₀₋₂-alkanediyl-OR⁷, C₀₋₂-alkandediyl-NH₂, C₀₋₂-alkanediyl-NHR⁷, C₀₋₂-alkanediyl-NR⁷R^{7′}, C₀₋₂-alkanediyl-NHCOR⁷, C₀₋₂-alkanediyl-NR⁷COR^{7′}, C₀₋₂-alkanediyl-NHSO₂R⁷, C₀₋₂-alkanediyl-NR⁷SO₂R^{7′}, C₀₋₂-alkanediyl-CO₂H, C₀₋₂-alkanediyl-CO₂R⁷,
C₀₋₂-alkanediyl-CONH₂, C₀₋₂-alkanediyl-CONHR⁷, C₀₋₂-alkanediyl-CONR⁷R^{7′}, phenyl and a five- or six-membered heteroaryl radical,
whereby the heteroaryl radical contains one or two heteroatoms, selected from the group that comprises N, S and O, whereby also the phenyl radical and the heteroaryl radical can be substituted with up to two radicals, selected from the group that comprises F, Cl, Br, CH₃, C₂H₅, OH, OCH₃, OC₂H₅, NO₂, N(CH₃)₂, CF₃, C₂F₅, and SO₂NH2 and/or also can carry an anellated methanediylbisoxy group or an ethane-1,2-diylbisoxy group,
whereby the piperazine radical on a second nitrogen atom can also be substituted with R⁷, COR⁷ or SO₂R⁷,
whereby R⁷ and R^{7′}, independently of one another, can be selected according to the meanings that are further indicated below,
R² means -Z-R^{2′}, an aryl group or a five- or six-membered heteroaryl group with one or two heteroatoms, selected from the group that comprises N, S and O, a benzothienyl group or an indolyl group, whereby the above-mentioned aryl or heteroaryl group can be substituted with up to three radicals, independently of one another, selected from the group that comprises
F, Cl, Br,
C(NH)NH₂, C(NH)NHR⁴, C(NH)NR⁴R^{4′}, C(NR⁴)NH₂, C(NR⁴)NHR^{4′},
C(NR⁴)NR⁴R^{4′},
X—OH, X—OR⁴, X—OCOR⁴, X—OCONHR⁴,
X—COR⁴, X—C(NOH)R⁴,
X—CN, X—COOH, X—COOR⁴, X—CONH₂, X—CONR⁴R^{4′}, X—CONHR⁴,
X—CONHOH,
X—SR⁴, X—SOR⁴, X—SO₂R⁴,
SO₂NH₂, SO₂NHR⁴, SO₂NR⁴R^{4′},
NO₂, X—NH₂, X—NHR⁴, X—NR⁴R⁴, X—NHSO₂R⁴, X—NR⁴SO₂R^{4′},
X—NHCOR⁴, X—NHCOOR⁴, X—NHCONHR⁴ and
R⁴,
whereby X is a bond, CH₂, (CH₂)₂, or CH(CH₃)₂,
whereby also radicals R⁴ and R^{4′} are selected independently of one another according to the meanings that are further indicated below, and
whereby two radicals at R¹, if they are in ortho-position to one another, can be linked to one another such that together they form a methanediylbisoxy, ethane-1,2-diylbisoxy-, propane-1,3-diyl or butane-1,4-diyl group,
Z means NH, NR^{2″}, O, S, SO or SO₂, whereby R″ has the meaning that is indicated below,
R^{2′} and R^{2″}, independently of one another, in each case mean a radical that is selected from the group that comprises:
C₁₋₄-perfluoroalkyl, C₁₋₆-alkyl, (C₀₋₃-alkanediyl-C₃₋₇ cycloalkyl),
(C₀₋₃-alkanediyl-aryl) and (C₀₋₃-alkanediyl-heteroaryl),
whereby the heteroaryl group is five- or six-membered and contains one or two heteroatoms, selected from the group that comprises N, S and O, and
whereby the aryl and heteroaryl group can be substituted in each case with up to two radicals, selected from the group that comprises F, Cl, Br, CH₃, C₂H₅, OH, OCH₃, OC₂H₅, NO₂, N(CH₃)₂, CF₃, C₂F₅, and SO₂NH₂ and/or also can carry an anellated methanediylbisoxy group or an ethane-1,2-diylbisoxy group, and
in addition in a five-membered cycloalkyl ring, a ring member can be ring N or ring O, and in a six- or seven-membered cycloalkyl ring, one or two ring members can be ring-N atoms and/or ring-O atoms, whereby the ring-N atoms optionally can be substituted with C₁₋₃-alkyl or C₁₋₃-alkanoyl,
or R^{2′} and R^{2″} together with Z form a five- to seven-membered heterocyclic ring, if Z is an N atom, whereby Z has the meaning that is further indicated above, whereby also the heterocyclic ring contains another N, O or S atom and optionally can be substituted with a radical that is selected from the group that comprises C₁₋₄-alkyl, (C₀₋₃-alkanediyl-C₁₋₃-alkoxy), C₁₋₄-alkanoyl, C₁₋₄-alkoxycarbonyl, aminocarbonyl and aryl,
R³, independently of one another, mean one or two radicals, selected from the group that comprises:
hydrogen,
F, Cl, Br,
OH, OR⁴, OCOR⁴, OCONHR⁴,
COR⁴,
CN, COOH, COOR⁴, CONH₂, CONHR⁴, CONR⁴R^{4′}, CONHOH,
CONHOR⁴,
SR⁴, SOR⁴, SO₂R⁴, SO₂NH₂, SO₂NHR⁴, SO₂NR⁴R^{4′},
NO₂, NH₂, NHR⁴, NR⁴R^{4′},
NHSO₂R⁴, NR⁴SO₂R^{4′}, NHSO₂R⁶, NR⁴SO₂R⁶,
NHCOR⁴, NHCOOR⁴, NHCONHR⁴ and R⁴,
whereby radicals R⁴, R^{4′} and R⁶ are selected independently of one another according to the meanings that are further indicated below,
A means a group that is selected from the group that comprises C₁₋₁₀-alkanediyl, C₂₋₁₀-alkenediyl, C₂₋₁₀-alkinediyl and (C₀₋₃-alkanediyl-C₃₋₇-cycloalkanediyl-C₀₋₃-alkanediyl),
whereby in a five-membered cycloalkyl ring, a ring member can be ring N or ring O, and in a six- or seven-membered cycloalkyl ring, one or two ring members in each case can be ring-N atoms and/or ring-O atoms,
whereby the ring-N atoms optionally can be substituted with C₁₋₃-alkyl or C₁₋₃-alkanoyl,
whereby in the above-mentioned aliphatic chains, a C atom can be exchanged for O, NH, N—C₁₋₃-alkyl or N—C ₁₋₃-alkanoyl and whereby alkyl or cycloalkyl groups optionally can be substituted with a radical that is selected from the group that comprises ═O, OH, O—C₁₋₃-alkyl, NH₂, NH—C₁₋₃-alkyl, NH—C₁₋₃-alkanoyl, N(C₁₋₃-alkyl)₂ and N(C₁₋₃-alkyl)(C₁₋₃-alkanoyl),
B means a radical that is selected from the group that comprises COOH, COOR⁵, CONH₂, CONHNH₂, CONHR⁵, CONR⁵R^{5′}, CONHOH, CONHOR⁵
and
tetrazolyl,
in each case bonded to a C atom of group A,
whereby radicals R⁵ and R^{5′}, independently of one another, are selected according to the meanings that are further indicated below,
Y means a group that is selected from the group that comprises O, NH, NR⁴, NCOR⁴, NSO₂R⁴ and NSO₂R⁶,
whereby R⁴ and R⁶ have the meanings that are further indicated below, in which in the radicals above, radicals R⁴, R^{4′}, R⁵, R^{5′} and R⁶ have the following meanings; here:
R⁴ and R^{4′}, independently of one another, in each case mean a radical that is selected from the group that comprises CF₃, C₂F₅, C₁₋₄-alkyl, C₂₋₄-alkenyl, C₂₋₃-alkinyl
and
(C₀₋₃-alkanediyl-C₃₋₇-cycloalkyl),
whereby in a five-membered cycloalkyl ring, a ring member can be ring N or ring O, and in a six- or seven-membered cycloalkyl ring, one or two ring members in each case can be ring-N atoms and/or ring-O atoms,
whereby the ring-N atoms optionally can be substituted with C₁₋₃-alkyl or C₁₋₃-alkanoyl,
R⁵ and R^{5′}, independently of one another, in each case mean a radical that is selected from the group that comprises C₁₋₆-alkyl, C₂₋₆-alkenyl, C₂₋₆-alkinyl, whereby a C atom can be exchanged for O, S, SO, SO₂, NH, N—C₁₋₃-alkyl or N—C₁₋₃-alkanoyl, and also (C₀₋₃-alkanediyl-C₃₋₇-cycloalkyl), whereby in a five-membered cycloalkyl ring, a ring member can be ring N or ring O, and in a six- or seven-membered cycloalkyl ring, one or two ring members in each case can be ring-N atoms and/or ring-O atoms, whereby the ring-N atoms optionally can be substituted with C₁₋₃-alkyl or C₁₋₃-alkanoyl, as well as also (C₀₋₃-alkanediyl-aryl) and (C₀₋₃-alkanediyl-heteroaryl), whereby the heteroaryl group is five- or six-membered and contains one or two heteroatoms that are selected from the group that comprises N, S and O,
whereby all above-mentioned alkyl and cycloalkyl radicals can be substituted with up to two radicals that are selected from the group that comprises CF₃, C₂F₅, OH, O—C₁₋₃-alkyl, NH₂, NH—C₁₋₃-alkyl, NH—C₁₋₃-alkanoyl, N(C₁₋₃-alkyl)₂, N(C₁₋₃-alkyl)(C₁₋₃-alkanoyl), COOH, CONH₂ and COO—C₁₋₃-alkyl, and all above-mentioned aryl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂ and/or also can carry an anellated methanediylbisoxy group or ethane-1,2-diylbisoxy group,
or R⁵ and R^{5′} together with the amide-N atom of B form a five- to seven-membered, saturated or unsaturated heterocyclic ring that can contain another N or O or S atom and that can be substituted with C₁₋₄-alkyl, (C₀₋₂-alkanediyl-C₁₋₄-alkoxy), C₁₋₄-alkoxycarbonyl, aminocarbonyl or aryl,
R⁶ means a radical that is selected from the group that comprises (C₀₋₃-alkanediyl-aryl) and (C₀₋₃-alkanediyl-heteroaryl), whereby the heteroaryl group is five- or six-membered and contains one or two heteroatoms that are selected from the group that comprises N, S and O, and whereby the aryl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂, and/or also can carry an anellated methanediylbisoxy group or an ethane-1,2-diylbisoxy group,
R⁷ and R^{7′}, independently of one another, mean R⁴ or R⁶, for the production of a pharmaceutical agent for treating a disease according to one of claims 1-5.

7. Use of a benzimidazole according to claim 6, characterized in that
R¹ means a phenyl group, which can be substituted with up to two radicals, independently of one another, selected from the group that comprises:
F, Cl, Br,
C(NH)NH₂, C(NH)NHR⁴, C(NH)NR⁴R^{4′}, C(R⁴)NH₂, C(NR⁴)NHR^{4′},
C(NR⁴)NR⁴R^{4′},
OH, OR⁴, OCOR⁴, OCONHR⁴,
COR, C(NOH)R⁴,
CN, COOH, COOR⁴, CONH₂, CONR⁴R^{4′}, CONHR⁴, CONHOH,
SR⁴, SOR⁴, SO₂R⁴,
SO₂NH₂, SO₂NHR⁴, SO₂NR⁴R^{4′},
NO₂, NH₂, NHR⁴, NR⁴R^{4′}, NHCONHR⁴ and
R⁴,
whereby radicals R⁴ and R^{4′} are selected independently of one another according to meanings that are indicated below and whereby two substituents at R¹ can be linked to one another such that together they form a methanediylbisoxy group, ethane-1,2-diylbisoxy group, propane-1,3-diyl group or butane-1,4-diyl group, if they are in ortho-position to one another,
R² means a monocyclic or bicyclic C₆₋₁₀-aryl group or a monocyclic or bicyclic 5- to 10-membered heteroaryl group with 1-2 heteroatoms, selected from the group that consists of N, S or O, whereby the above-mentioned aryl group or heteroaryl group can be substituted with up to three of the following substituents, independently of one another:
F, Cl, Br,
XOH, XOR⁴, XOCOR⁴, XOCONHR⁴, XOCOOR⁴,
XCOR⁴, XC(NOH)R⁴, XC(NOR⁴), XC(NO(COR⁴))R⁴,
XCOOH, XCOOR⁴, XCONH₂, XCONHR⁴, XCONR⁴R⁴, XCONHOH,
XCONHOR⁴, XCOSR⁴,
XSR⁴, XSOR⁴, XSO₂R⁴, SO₂NH₂, SO₂NHR⁴, SO₂NR⁴R⁴,
NO₂, XNHR⁴, XNR⁴R⁴, XNHSO₂R⁴, XN(SO₂R⁴) SO₂R⁴, XNR⁴SO₂R⁴ and
R⁴,
whereby two substituents at R², if they are in ortho-position to one another, can be linked to one another such that together they form methanediylbisoxy, ethane-1,2-diylbisoxy, propane-1,3-diyl, butane-1,4-diyl,
R³ means a radical that is selected from the group that comprises hydrogen, F, Cl, Br, CH₃, C₂H₅, CF₃, C₂F₅, OH, OR⁴, NHSO₂R⁶ and NHCOR⁴,
whereby R⁴ and R⁶ have the meanings that are further indicated below,
A means C₁₋₁₀-alkanediyl, C₂₋₁₀-alkenediyl, C₂₋₁₀-alkinediyl, (C₀₋₅-alkanediyl-C₃₋₇-cycloalkanediyl-C₀₋₅-alkanediyl),
whereby in a 5-membered cycloalkyl ring, a ring member can be an N or an O, and in a 6- or 7-membered cycloalkyl ring, one or two ring members can be N and/or O, whereby ring nitrogens optionally can be substituted with C₁₋₃-alkyl or C₁₋₃-alkanoyl,
whereby in the above-mentioned aliphatic chains, a carbon atom or two carbon atoms can be exchanged for O, NH, NC₁₋₃-alkyl, or NC₁₋₃-alkanoyl,
B means a radical that is selected from the group that comprises COOH, COOR⁵, CONH₂, CONHR⁵ and CONR⁵R^{5′}, in each case bonded to a C atom of group A,
whereby radicals R⁵ and R^{5′} can be selected independently of one another according to the meanings that are further indicated below,
Y means O,
in which in the above radicals, radicals R⁴, R^{4′}, R⁵, R^{5′} and R⁶ have the following meanings; here:
R⁴ and R^{4′} have the same meaning as further indicated above,
R⁵ and R^{5′}, independently of one another, in each case mean a radical that is selected from the group that comprises C₁₋₆-alkyl, C₂₋₆-alkenyl, C₂₋₆-alkinyl, whereby a C atom can be exchanged for O, S, SO, SO₂, NH, N—C₁₋₃-alkyl or N—C₁₋₃-alkanoyl, also (C₀₋₃-alkanediyl-C₃₋₇-cycloalkyl),
whereby in a five-membered cycloalkyl ring, a ring member can be ring N or ring O, and in a six- or seven-membered cycloalkyl ring, one or two ring members in each case can be ring-N atoms and/or ring-O atoms,
whereby the ring-N atoms optionally can be substituted with C₁₋₃-alkyl or C₁₋₃-alkanoyl, as well as also (C₀₋₃-alkanediyl-phenyl) and (C₀₋₃-alkanediyl-heteroaryl), whereby the heteroaryl group is five- or six-membered and contains one or two heteroatoms that are selected from the group that comprises N, S and O,
whereby all above-mentioned alkyl and cycloalkyl radicals can be substituted with a radical that is selected from the group that comprises CF₃, C₂F₅, OH, O—C₁₋₃-alkyl, NH₂, NH—C₁₋₃-alkyl, NH—C₁₋₃-alkanoyl, N(C₁₋₃-alkyl)₂, N(C₁₋₃-alkyl)(C₁₋₃-alkanoyl), COOH, CONH₂ and COO—C₁₋₃-alkyl, and all above-mentioned phenyl and heteroaryl groups can be substituted with up to two radicals, selected from the group that comprises F, Cl, Br, CH₃, C₂H₅, OH, OCH₃, OC₂H₅, NO₂, N(CH₃)₂, CF₃, C₂F₅ and SO₂NH₂ and/or also can carry an anellated methanediylbisoxy group or ethane-1,2-diylbisoxy group,
or R⁵ and R^{5′} together with the amide-N atom of B form a five- to seven-membered, saturated or unsaturated heterocyclic ring that can contain another N or O or S atom and that can be substituted with C₁₋₄-alkyl, (C₀₋₂-alkanediyl-C₁₋₄-alkoxy), C₁₋₄-alkoxycarbonyl, aminocarbonyl or phenyl,
R⁶ means a phenyl or heteroaryl group, whereby the heteroaryl group is five- or six-membered and contains one or two heteroatoms that are selected from the group that comprises N, S and O, and whereby the phenyl and heteroaryl groups can be substituted with up to two radicals that are selected from the group that comprises F, Cl, Br, CH₃, C₂H₅, OH, OCH₃, OC₂H₅, NO₂, N(CH₃)₂, CF₃, C₂F₅ and SO₂NH₂, or else can carry an anellated methanediylbisoxy group or ethane-1,2-diylbisoxy group.

8. Use of a benzimidazole according to claim 6 or 7, wherein R³ is hydrogen and Y-A is C₁₋₆-alkylenoxy.
